SEQUENCE LISTING (3 sequences)

SEQ ID NO: 1 - DNA, 600 bp, Artificial Sequence
  Nucleotide sequence of KRT26 gene
cttcccagcccacgagtaggacgctggatgggaagaggagcagccagaacttgaacaacaactcggacatagggtttgggagaagcaaaccgcagcttagctccctgggacagatgaaagcatacaccgccattgcactttgcatattcacagccttccttggttcctctcacagaaagagtcctacgagagctccttggcggagatggaaggaaattactgcctccagctccagcaaatccaggatcagatcggggccatggaggggcagctgcagcagatccggacggaaaccgccggccagaagctggagcacgagcagctgctagacatcaaagtcttcttagagaaggagatcgagacgtactgcaacttactcgacggccaagacaggtgagccacccgccctcacgtcagccaggctttctccgcgtgtttcccgagagcgcgtcctagaatttcctgctaggaggatcacgccccagaggtcctcactcacgtactgcggcgcgtcgcagtcaaagcagagcacagtctagaaagtacccgtatgcatcctaatataaaggaggcttgtttagaaacaggatgtctgccggt

SEQ ID NO: 2 - DNA, 20 bp, Artificial Sequence
  Primer for amplifying the KRT26 gene
aaagagtcctacgagagctc

SEQ ID NO: 3 - DNA, 20 bp, Artificial Sequence
  Primer for amplifying the KRT26 gene
ctgtcttggccgtcgagtaa

(19) United States
(12) Patent Application Publication
(10) Pub. No.: US 2022/0186289 A1
(43) Pub. Date: Jun. 16, 2022

(54) MOLECULAR MARKER RELATED TO WOOL YIELD OF LONG-HAIRED RABBIT AND USE THEREOF

(71) Applicants: Shandong Agricultural University, Tai'an City (CN); Mengyin Yida Rabbit Industry Co., Ltd, Linyi City (CN)

(72) Inventors: Xinzhong FAN, Tai'an City (CN); Liang SONG, Tai'an City (CN); Xibo QIAO, Tai'an City (CN); Aiguo YANG, Tai'an City (CN); Yuanfeng YANG, Tai'an City (CN); Lu ZHANG, Tai'an City (CN); Zhaolu WANG, Tai'an City (CN)

(73) Assignees: Shandong Agricultural University, Tai'an City (CN); Mengyin Yida Rabbit Industry Co., Ltd, Linyi City (CN)

(21) Appl. No.: 17/545,789

(22) Filed: Dec. 8, 2021

(30) Foreign Application Priority Data
Dec. 16, 2020 (CN) 202011500756.4

(51) Int. Cl. C12Q 1/686; C12Q 1/6876

(52) U.S. Cl. CPC C12Q 1/686; C12Q 2600/156; C12Q 1/6876

(57) ABSTRACT

The present disclosure relates to the technical field of molecular marker breeding of rabbits, in particular to a molecular marker related to a wool yield of a long-haired rabbit and use thereof in breeding. The molecular marker includes a mutant of a keratin 26 gene (KRT26 gene), where the KRT26 gene is as shown in SEQ ID NO: 1, the mutant of the KRT26 gene is formed by mutation of a base G at position 41844263 of a KRT26 gene locus to a base A. In the present disclosure, the wool yield trait of the long-haired rabbit and the KRT26 gene are subjected to association study and population verification, and it is found that an individual long-haired rabbit with an allele G has a higher wool yield, with an additive gene effect of 15.59 g; the base substitution can control an overall genetic variation of the wool yield by 1.51%.

### CROSS REFERENCE TO RELATED APPLICATIONS)

This patent application claims the benefit and priority of Chinese Patent Application No. 202011500756.4, filed on Dec. 16, 2020, the disclosure of which is incorporated by reference herein in its entirety as part of the present application.

### TECHNICAL FIELD

The present disclosure relates to the technical field of molecular marker breeding of rabbits, in particular to a molecular marker related to a wool yield of a long-haired rabbit and use thereof in breeding.

### BACKGROUND ART

Rabbit wool, due to slender and soft fibers, fluffy and light texture, and desirable warmth retention, is a high-grade textile material with excellent texture and occupies an important position in the textile industry. With the improvement of wool spinning technology and the development of new products, the demands have increased rapidly for rabbit hair/wool in domestic and oversea markets. China has the largest number of breeding for long-haired rabbits in the world, but an individual long-haired rabbit has a relatively low wool yield. Therefore, it is of great production significance to breed long-haired rabbits with a high wool yield.

Conventional breeding methods to increase the wool yield of long-haired rabbits are based on phenotypic selection. That is, the wool yield of a candidate group is measured, a breeding value of a phenotypic value of candidate individuals and their relatives are estimated using statistical genetics methods, and environmental effects are eliminated, such that individuals with high breeding values are selected as a seed stock. However, the conventional breeding methods have a relatively low accuracy of selection affected by the scale and standardization of performance measurement, and the genetic evaluation methods. Moreover, when selecting to increase the wool yield, the conventional breeding methods is difficult to take into account the relevant selection responses to the quality traits of rabbit wool. This may generally leads to a larger diameter of the rabbit wool fiber, a decreased textile performance and a decreased value of wool spinning after the wool yield is increased.

Wool fiber is mainly composed of keratin (KRT) and keratin-associated protein (KAP), where the keratin can affect hair diameter, and participate in the differentiation of hair structures and the formation of skin appendages. Keratin 26 gene (KRT26 gene) can encode a protein of 468 amino acids. As a special type-1 keratin, The KRT26 is specifically expressed in an internal root sheath of hair follicles and mainly regulates growth and development of the entire hair follicles. Yuezhen Tian et al. have found that a superfine wool sheep has an expression level of the KRT25 gene 1.65 times than that of a fine wool sheep.

Single nucleotide polymorphism (SNP) markers mainly refer to DNA sequence polymorphisms caused by single nucleotide variations at the genome level. The SNP is the most common type of heritable variations in all organisms, accounting for not less than 90% of all known polymorphisms. At present, researches using genome resequencing technology or high-density SNP chip technology are based on genome-wide SNP site detection as a goal. The genome wide association study (GWAS) technology developed from these researches has greatly promoted the efficiency of SNP site screening and the accuracy of prediction results. This technology is particularly effective in the field of mining functional genes or functional sites for complex traits controlled by minor polygenes. It is one of the effective methods to improve the yield and quality of rabbit wool by studying candidate genes and rabbit wool growth-related molecular markers from the perspective of molecular biology. The method can provide a reference and basis for long-haired rabbit breeding.

### SUMMARY

To overcome the above shortcomings of the prior art, the present disclosure provides a molecular marker related to a wool yield of a long-haired rabbit. Meanwhile, the present disclosure further provides use of the molecular marker related to a wool yield of a long-haired rabbit.

To achieve the above objective, the present disclosure adopts the following technical solutions.

A molecular marker related to a wool yield of a long-haired rabbit includes a mutant of a KRT26 gene, where the KRT26 gene is as shown in SEQ ID NO: 1, and the mutant of the KRT26 gene is formed by mutation of a base G at position 41844263 of a KRT26 gene locus to a base A.

In the present disclosure, the base G at the position 41844263 of the KRT26 gene locus in the long-haired rabbit is mutated to the base A to form the mutant; a long-haired rabbit with an allele G has a higher wool yield, while a long-haired rabbit with an allele A has a lower wool yield. Therefore, in breeding work, if a gene frequency of the allele G in a long-haired rabbit population is increased to establish a GG homozygous population, the wool yield of the long-haired rabbit population can be increased.

The present disclosure further provides an amplification primer for detecting the molecular marker. The amplification primer includes primers as shown in SEQ ID NO: 2 and SEQ ID NO: 3, for amplifying the KRT26 gene.

The present disclosure further provides use of the molecular markers in selection of a wool yield trait in the long-haired rabbit.

The present disclosure further provides a screening method of the molecular marker, including the following steps: conducting polymerase chain reaction (PCR) amplification on a genomic DNA of an individual sample of a long-haired rabbit to be tested using the primers to obtain PCR products, selecting a gene SNP site based on the PCR products, and conducting SNP typing using a flight mass spectrometry method to determine whether a base at position 41844263 of a KRT26 gene locus is a base G or mutated to a base A.

Preferably, the PCR products may have a length of 250 bp.

Preferably, the SNP typing using a flight mass spectrometry method may specifically include the following steps:

S1, according to SNP site information, designing PCR reaction and single-base amplification primers, and conducting quality control on genomic DNA samples to obtain qualified genomic DNA samples;

S2, subjecting the qualified genomic DNA samples to PCR reaction, and conducting SAP digestion and extension to obtain a reaction product; and

S3, diluting the reaction product, desalting by a resin, spotting a desalted sample on a sample target, crystallizing naturally, and conducting mass spectrometry detection to collect data.

Preferably, the selecting a gene SNP site may be conducted by DNA pooling sequencing, specifically including the following steps:

S1, DNA pooling construction: randomly selecting 100 genomic DNA samples from individuals of long-haired rabbits to be tested, and mixing each 20 genomic DNA samples in equal volume into a DNA pooling; and

S2, according to a rabbit KRT26 gene sequence on a database, conducting PCR amplification with the primers at an annealing temperature of 45-55° C.

Preferably, in step S2, the annealing temperature may be 53° C.

In the present disclosure, the SNP typing is conducted using the SNP site existing in the KRT26 gene by a flight mass spectrometry method. Through sequence comparison, it is found that the base G at the position 41844263 of the KRT26 gene locus is mutated to the base A. It is revealed by association analysis on the hair yield trait and the KRT26 gene of the long-haired rabbit that, three genotypes formed by the SNP site of the KRT26 gene are closely associated to the wool yield trait of the long-haired rabbit. A GG type has a wool yield extremely significantly higher than that of AA and AG types. An additive gene effect of the GG is 15.59 g, such that this base substitution can control an overall genetic variation of wool yield by 1.51%. Therefore, the wool yield of the long-haired rabbit can be improved by increasing the gene frequency of the allele G and/or reducing the gene frequency of the allele A in the long-haired rabbit population. The base mutation at the position 41844263 of the KRT26 gene locus provides a molecular marker with breeding value for the selection of the wool yield trait in the long-haired rabbit.

Compared with the prior art, the present disclosure has the following beneficial effects:

In the present disclosure, it is discovered for the first time that the base G at the position 41844263 of the KRT26 gene locus in the long-haired rabbit is mutated to the base A; the long-haired rabbit with the allele G has a higher wool yield, while the long-haired rabbit with the allele A has a lower wool yield. In the breeding of long-haired rabbit, the gene frequency of the allele G in the long-haired rabbit population is increased to significantly increase the wool yield of the long-haired rabbit. This can provide a basis for further selection of long-haired rabbit breeds with a high wool yield.

### BRIEF DESCRIPTION OF THE DRAWINGS

FIG. 1 shows a schematic diagram of detection results of agarose gel electrophoresis for a PCR amplified product of the present disclosure;

FIG. 2 shows a schematic diagram of results of cloning sequencing of a PCR amplified product of a KRT26 gene sequence of the present disclosure; and

FIG. 3 shows a gene sequencing peak diagram of the PCR amplified product of the KRT26 gene sequence of the present disclosure.

### DETAILED DESCRIPTION OF THE EMBODIMENTS

To better explain the objectives, technical solutions, and advantages of the present disclosure, the present disclosure will be further explained below with reference to accompanying drawings and specific examples. It should be emphasized that, unless otherwise specified, the technical means used in these examples are conventional means well known to those skilled in the art, and the reagents used are all commercially-available chemical reagents.

Example I Detection of a KRT26 gene mutant in a long-haired rabbit

1. Selection of test materials: in the present disclosure, 757 long-haired rabbit samples in total were used as test materials, which were jointly bred by Shandong Mengyin Vida Rabbit Industry Co., Ltd. and Shandong Agricultural University. On the 283th day after birth (fourth shearing), the rabbits were raised for wool growth under uniform conditions for 73 d, and an individual wool yield and fiber quality indicators were measured. Wool samples were cut on a center of a side of the rabbit body using scissors, a piece of ear tissue with a size of a soybean grain was cut using surgical scissors; the ear tissue was put into a 1.5 mL centrifuge tube containing 75% alcohol, and taken back to the laboratory, stored at −20° C. for subsequent genomic DNA extraction. The reagents used in the experiment were purchased from companies such as TaKaRa.

2. Experimental method: a genomic DNA was extracted from the ear tissue sample by high-salt method, and DNA concentration and quality were detected with a spectrophotometer. The extracted DNA was stored at -20° C.

S1. DNA pooling construction: 100 genomic DNA samples were randomly selected from the above individuals of long-haired rabbits to be tested, and each 20 genomic DNA samples were mixed in equal volume into a DNA pooling.

S2. Candidate gene primer design: according to a rabbit KRT26 gene sequence on an ensemble database (such as SEQ ID NO: 1), primers of an exon coding region of the above gene was designed using Primer 5.0, where the primers were synthesized by Sangon Biotech (Shanghai) Co., Ltd., and a primer sequence information was shown in the table below.

TABLE 1
Primers for amplifying genomic DNA

| | | | Annealing |
|---|---|---|---|
| Primer | | Length | temperature |
| name | Sequence (5′-′) | bp | (° C.) |
| KRT26 | F AAAGAGTCCTACGAGAGCTC | 250 | 53.0 |
| | R CTGTCTTGGCCGTCGAGTAA | | |

the following components were added in to a PCR tube:

| | |
|---|---|
| KRT26 gene sequence (50 μg/ml) | 1.0 μL |
| Primer F (10 μmol/ml) | 1.0 μL |
| Primer R (10 μmol/ml) | 1.0 μL |
| Taq enzyme mixed solution | 12.5 μL |
| adding ddH₂O to make up to: | 25 μL |

PCR amplification is conducted by:
pre-denaturation at 95° C. for 10 min;
conducting 35 cycles: denaturation at 95° C. for 45 sec
annealing at 53° C.
extension at 72° C. for 30 sec;
extension at 72° C. for 10 min

After the reaction was complete, PCR products were stored in a refrigerator at 4° C. for sample loading detection and subsequent test analysis.

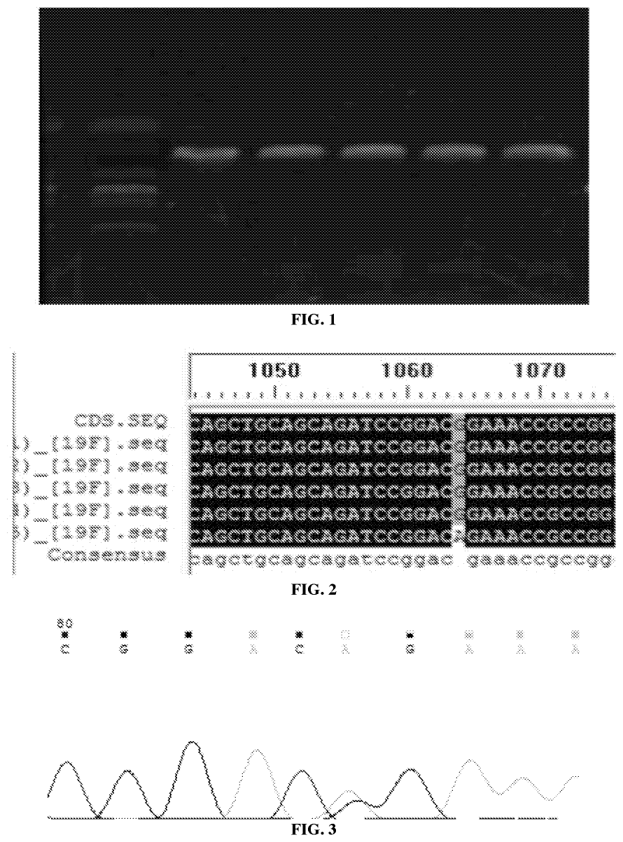

PCR amplification results were detected using 1% agarose gel electrophoresis. Electrophoresis detection shows that the KRT26 gene sequence is specific to all PCR products of the primer, and a fragment size is consistent with expectations, and there is no non-specific amplified band (referring to the electrophoresis diagram in FIG. 1).

The qualified PCR products were sent to Sangon Biotech (Shanghai) Co., Ltd. for cloning sequencing, and gene SNP sites were selected; SNP typing was conducted by a flight mass spectrometry method; after the cloning sequencing was completed, sequencing results were analyzed using a DNAMAN software and a Chrotnas software to find SNPs. The results are shown in FIG. 2:

Through sequence alignment, it was found that a base G at position 41844263 of the KRT26 gene locus was mutated to a base A, or a mutation point was selected using a peak map. The result is shown in FIG. 3, different peaks appear at a same sequence site, indicating that base mutation has occurred at this site, and a double peak indicates a heterozygote.

### Example 2 SNP Typing

SNP typing was conducted using flight mass spectrometry by Beijing Compass Biotechnology Co., Ltd., and specific steps were as follows:

(1) Primer design: according to SNP site information, a PCR reaction and single-base extension primers were designed using a software AssayDesignSuitev2.0 designed by MassARRAY, and a specificity of primers was tested online through University of California-Santa Cruz (UCSC) Genome Browser.

(2) Genomic DNA quality inspection: DNA concentration, purity and degree of degradation were detected by agarose gel electrophoresis, where an interpretation standard of test results was: in a gel image of the electrophoresis, the DNA band was single, clear, free of impurities, and there was no dispersion or tailing.

(3) Electrophoresis conditions:

1) 0.8% agarose gel. 170V and 25 min,

2) sample loading volume: 500 ng of a sample+3 μl of a Loading Buffer; and

3) Marker: 3 μl of a Trans2000 Plus.

(4) PCR reaction:

1) A PCR reaction system was shown in Table 2 below:

TABLE 2
PCR reaction system

| Reagent | Concentration | | Volume(μl) |
|---|---|---|---|
| Water, HPLCgrade | NA | | 927.5 |
| PCRBufferwith15mMMgCl2 | 10 x | | 331.25 |
| MgCl2 | 25 | mM | 172.25 |
| dNTPMix | 25 | mM | 53 |
| PrimerMix | 0.5 | uM | 530 |
| HotStarTaq | 5 | U/μl | 106 |
| DNAtemplate | 10 | ng/μl | 1/well |

| | |
|---|---|
| Total | 5/well |

2) Cycle parameters of the PCR reaction were shown in Table 3 below:

TABLE 3
PCR reaction cycle parameters

| Temperature (° C.) | Time(second) | Cycle |
|---|---|---|
| 94 | 120 | 1 |
| 94 | 20 | |
| 56 | 30 | 45 |
| 72 | 60 | |
| 72 | 180 | 1 |
| 4 | ∞ | 1 |

(5) SAP digestion

1) A SAP digestion reaction system was shown in Table 4:

TABLE 4
SAP digestion reaction system

| Reagent | Concentration | Volume(ul) |
|---|---|---|
| Water | NA | 810.9 |
| SAPBUffer | 10 x | 90.1 |
| SAP | 1.7 U/ul | 159.0 |
| Total | | 2/well |

2) Cycle parameters of the SAP digestion were shown in Table 5:

TABLE 5
SAP digestion cycle parameters

| Temperature(° C.) | Time(minute) | Cycle |
|---|---|---|
| 37 | 40 | 1 |
| 85 | 5 | 1 |
| 4 | ∞ | 1 |

(6) Extension reaction

1) An extension reaction system was shown in Table 6 below:

TABLE 6
Extension reaction system

| Reagent | Concentration | Volume(ul) |
|---|---|---|
| Water | NA | 400.2 |
| iPLEXbufferplus | 10x | 106 |
| iPLEXterminator | NA | 106 |
| PrimerMix | 0.6-1.3 uM | 426.1 |
| iPlexenzyme | NA | 21.7 |
| total | | 2/well |

2) Cycle parameters of the extension reaction were shown in Table 7 below:

TABLE 7
Extension reaction cycle parameters

| Temperature (° C.) | Time (sec) | Cycle |
|---|---|---|
| 94 | 30 | 1 |
| 94 | 5 | 1 |
| 52 | 5 | |
| 80 | 5 | 40 |
| 72 | 180 | 1 |
| 4 | ∞ | |

(7) Detection on machines:

1) the reaction product (9 ul in total) was diluted by 3 times and desalted using a resin;

2) a desalted sample was spotted on a sample target and crystallized naturally; and

3) mass spectrometry detection was conducted to collect data.

According to typing results, population genetic analysis of SNPs and an association analysis of the SNPs with a wool fiber diameter trait of the long-haired rabbit were conducted using an R software and a SAS software.

Association analysis of the examples, the wool yield trait of the long-haired rabbit and the KR 126 gene

An analysis of variance was conducted by a general linear model (GLM) procedure using SAS9.2, and an association analysis between each genotype of the polymorphic sites and the wool yield trait of the long-haired rabbit was conducted. A best linear unbiased predictor (BLUP) model was:

Y=Xb+Za+e

Y: a phenotypic value of wool production trait; X: an individual number matrix related to a fixed effect; b: a fixed effect (SNP site, and gender effect); Z: an individual number matrix of an individual additive genetic effect; a: individual additive genetic effect; and e: a random error; according to KRT26 gene SNPs, allele and genotype frequencies were calculated separately, and genotypes AA, AG and GG were obtained. Association analysis results of each genotype corresponding to the wool yield were shown in Table 8.

TABLE 8
Association analysis results of KRT26 gene and
wool yield trait of long-haired rabbit

| | Genotype | | | |
|---|---|---|---|---|
| Trait | AA (596) | AG (85) | GG (76) | P |
| Wool yield | 300.79 ± 2.65^{B} | 313.53 ± 6.86^{b} | 330.86 ± 9.02^{A} | <0.05 |

From the data in Table 8, it can be shown that the position 41844263 of KRT26 gene locus has a significant effect on the wool yield trait of the long-haired rabbit (P<0.05), where a. wool yield of a genotype GG long-haired rabbit is 30.07 g and 17.33 g higher than that of genotype AA and genotype AG long-haired rabbits, respectively. An additive gene effect of the GG is 15.59 g, such that this base substitution can control an overall genetic variation of wool yield by 1.51%.

The base G at the position 41844263 of the KRT26 gene locus in the long-haired rabbit is mutated to the base A; a long-haired rabbit with an allele G has a higher wool yield, while a long-haired rabbit with an allele A has a lower wool yield. Therefore, in breeding work, the gene frequency of the allele G in a long-haired rabbit population is increased by marker-assisted selection, to establish an allele G homozygous population, thereby significantly increasing the wool yield of the long-haired rabbit.

Finally, it should be noted that the above examples are provided merely to describe the technical solutions of the present disclosure, rather than to limit the protection scope of the present disclosure. Although the present disclosure is described in detail with reference to preferred examples, a person of ordinary skill in the art should understand that modifications or equivalent replacements may be made to the technical solutions of the present disclosure without departing from the spirit and scope of the technical solutions of the present disclosure.

1. A molecular marker related to a wool yield of a long-haired rabbit, comprising a mutant of a keratin 26 gene (KRT26 gene), wherein the KRT26 gene is as shown in SEQ ID NO: 1, and the mutant of the KRT26 gene is formed by mutation of a base G at position 41844263 of a KRT26 gene locus to a base A.

2. An amplification primer of the molecular marker according to claim 1, comprising primers as shown in SEQ ID NO: 2 and SEQ ID NO: 3, for amplifying the KRT26 gene.

3. (canceled)

4. A screening method of the molecular marker according to claim 1, comprising the following steps: conducting polymerase chain reaction (PCR) amplification on a genomic DNA of an individual sample of a long-haired rabbit to be tested using the primers according to claim 2 to obtain PCR products, selecting a gene single nucleotide polymorphism (SNP) site based on the PCR products, and conducting SNP typing using a flight mass spectrometry method to determine whether a base at position 41844263 of a KRT26 gene locus is a base G or mutated to a base A.

5. The screening method according to claim 4, wherein the PCR products have a length of 250 bp.

6. The screening method according to claim 4, wherein the SNP typing using a flight mass spectrometry method specifically comprises the following steps:
S1, according to SNP site information, designing PCR reaction and single-base amplification primers, and conducting quality control on genomic DNA samples to obtain qualified genomic DNA samples;
S2, subjecting the qualified genomic DNA samples to PCR reaction, and conducting SAP digestion and extension to obtain a reaction product; and
S3, diluting the reaction product, desalting by a resin, spotting a desalted sample on a sample target, crystallizing naturally, and conducting mass spectrometry detection to collect data.

7. The screening method according to claim 4, wherein the selecting a gene SNP site is conducted by DNA pooling sequencing, specifically comprising the following steps:
S1, DNA pooling construction: randomly selecting 100 genomic DNA samples from individuals of long-haired rabbits to be tested, and mixing each 20 genomic DNA samples in equal volume into a DNA pooling; and
S2, according to a rabbit KRT26 gene sequence on a database, conducting PCR amplification with the primers according to claim 2 at an annealing temperature of 45-55° C.

8. The screening method according to claim 7, wherein in step S2, the annealing temperature is 53° C.
